(19) United States
(12) Patent Application Publication
(10) Pub. No.: US 2025/0256092 A1
(43) Pub. Date: Aug. 14, 2025

(54) Conductive Kinesiology Tape

(71) Applicant: Lectra Technologies LLC, Camp Hill, PA (US)

(72) Inventors: Jacob D. Henry, Camp Hill, PA (US); Nicholas P. Squitieri, Ridgefield, CT (US)

(73) Assignee: Lectra Technologies LLC, Camp Hill, PA (US)

(21) Appl. No.: 19/110,624

(22) PCT Filed: Oct. 27, 2023
(86) PCT No.: PCT/US2023/036093

§ 371 Date: Mar. 11, 2025

Related U.S. Application Data
(60) Provisional application No. 63/381,227, filed on Oct. 27, 2022.

(51) Int. Cl. A61N 1/04; A61F 13/00; A61F 13/0246; A61N 1/02; A61N 1/36

(52) U.S. Cl. CPC A61N 1/0484; A61F 13/00063; A61F 13/0246; A61N 1/025; A61N 1/3603; A61F 2013/00604; A61F 2013/00663; A61F 2013/0071; A61N 1/0452; A61N 1/0456

(57) ABSTRACT

A conductive kinesiology tape includes an elastic fabric having a bottom surface and a top surface with an adhesive layer coupled to the bottom surface. The conductive kinesiology tape also includes conductive threads incorporated into the elastic fabric such that a portion of each conductive thread is exposed on the top surface of the elastic fabric and a portion of each conductive thread is exposed on the bottom surface of the elastic fabric.

### CROSS-REFERENCE TO RELATED APPLICATIONS

This application claims the benefit of and priority to U.S. Provisional Patent Application No. 63/381,227, filed Oct. 27, 2022, which is incorporated herein by reference in its entirety.

### FIELD OF THE DISCLOSURE

The disclosure provides a conductive kinesiology tape and its therapeutic use.

### BACKGROUND OF THE DISCLOSURE

Kinesiology tape or Kinesio® tape was developed by Dr. Kenzo Kase in the 1970s to provide support without limiting movement. The original kinesiology tape of Dr. Kase was made from a highly elastic cotton/nylon blend to make the tape extremely flexible, with hypoallergenic adhesive to keep the tape in place without causing a rash. Today there are at least 50 brands of kinesiology tape commercially available, based on Dr. Kase's original design.

Kinesiology tape is stretched before application and when it is applied, it recoils slightly, gently lifting the skin. The resulting gap between the skin and underneath tissue may reduce pressure on the tissue and thereby reduce pain. Kinesiology tape can also improve blood flow in the skin and the subcutaneous gap created may lead to improved circulation of lymphatic fluids, which may reduce swelling and improve healing.

Electrical stimulation or electrostimulation of nerve fibers and muscle fibers provides benefits complementary to kinesiology tape. Transcutaneous electrical nerve stimulation (TENS) transmits low voltage electrical impulses through the skin to provide pain relief. It is believed that TENS operates by stimulating nerve fibers that either block the transmission of pain signals or raise the level of pain-reducing endorphins. A typical TENS unit consists of a handheld or tabletop electrical impulse generating device that is connected by wires to two electrode pads that are attached to the skin at desired locations.

Electronic muscle stimulation (EMS) is similar to TENS but the electrical impulse is stronger and triggers repeated muscle contractions. The electrical signal can be adjusted to provide short, frequent contractions or long, sustained ones. EMS therapy encourages blood circulation, muscle stimulation, and healing, and can be used to improve and maintain muscle tone in weak or atrophied muscles. EMS can also be used to increase blood flow and reduce inflammation in joints. Typical EMS units are similar to TENS units, consisting of an electrical impulse generator attached by wires to electrode pads that are attached to the desired skin location.

A need exists for an enhanced kinesiology tape that incorporates portable, hands-free electrostimulation without sacrificing the elasticity, breathability, comfort and function of the kinesiology tape. The present conductive kinesiology tape meets this critical need.

### SUMMARY OF THE DISCLOSURE

In a first aspect of the disclosure, a conductive kinesiology tape is provided. The tap includes an elastic fabric having a bottom surface and a top surface. The conductive kinesiology tape also includes conductive threads incorporated into the elastic fabric such that a portion of each conductive thread is exposed on the top surface of the elastic fabric, and a portion of each conductive thread is exposed on the bottom surface of the elastic fabric. The conductive kinesiology tape further includes an adhesive layer coupled to the bottom surface.

In a second aspect of the disclosure the conductive kinesiology tape comprising a first layer and a second layer. The first layer includes an elastic fabric having a bottom surface and a top surface opposite the bottom surface. The first layer further includes a first conductive thread incorporated into the elastic fabric such that a portion of the first conductive thread is exposed on the top surface of the elastic fabric to form a positive conductive area, and a portion of the first conductive thread is exposed on the bottom surface of the elastic fabric to form a positive electrode. The first layer also includes a second conductive thread incorporated into the elastic fabric such that a portion of the second conductive thread is exposed on the top surface of the elastic fabric to form a negative conductive area, and a portion of the second conductive thread is exposed on the bottom surface of the elastic fabric to form a negative electrode. The second layer includes an adhesive disposed on the bottom surface of the elastic fabric.

In certain embodiments, the elastic fabric comprises elastic cotton and nylon. In other embodiments, the conductive threads are incorporated into the elastic fabric in a zigzag pattern. In certain embodiments, the conductive threads are woven or stitched into the elastic fabric. In certain embodiments, the conductive threads comprise at least one member chosen from carbon nanotubes, metallized thread, conductive polymer nanofibers, and reduced graphene. In certain embodiments, the conductive threads comprise carbon nanotubes. In certain embodiments, the conductive threads comprise metallized thread.

In certain embodiments, the adhesive comprises a pressure-sensitive adhesive. In certain embodiments, the adhesive comprises an acrylic polymer. In certain embodiments, the adhesive comprises a methacrylate polymer or epoxy diacrylate polymer. In certain embodiments, the adhesive further comprises a therapeutic agent. In certain embodiments, the adhesive further comprises a sustained release therapeutic agent. In certain embodiments, the therapeutic agent comprises at least one member chosen from menthol, methyl salicylate, and cannabidiol.

In certain embodiments, the conductive kinesiology tape comprises no other layers. In other words, in these embodiments, the conductive kinesiology tape consists of the first and second layers. In certain embodiments, the conductive kinesiology tape does not comprise a thermoplastic polymer. In other words, in these embodiments, the conductive kinesiology tape is free of thermoplastic polymers. In certain embodiments, the conductive kinesiology tape is free of conductive ink.

These and other aspects of the conductive kinesiology tape will be apparent upon reference to the following description. To this end, various references are set forth herein which describe in more detail certain background information, procedures, compounds, and/or compositions, and are each hereby incorporated by reference in their entirety.

### BRIEF DESCRIPTION OF THE DRAWINGS

Advantages of the present invention will be readily appreciated, as the same becomes better understood by reference to the following detailed description, when considered in connection with the accompanying drawings.

FIG. 1A is a top view of the conductive kinesiology tape with elastic fabric, removable masking layer, and pockets.

FIG. 1B is a top view of the conductive kinesiology tape with elastic fabric, conductive thread, positive and negative conductive areas, and pockets.

FIG. 2A is a bottom view of the conductive kinesiology tape with elastic fabric and positive and negative electrodes.

FIG. 2B is a bottom view of the conductive kinesiology tape with elastic fabric, conductive thread, and positive and negative electrodes.

FIG. 3 shows a cross-sectional side view of the conductive kinesiology tape with clastic fabric, conductive threads, positive and negative conductive areas, positive and negative electrodes, and pockets.

FIG. 4A is a side view of a wireless control unit with a housing along with positive and negative conductive elements.

FIG. 4B is a side view of a wireless control unit with housing along with positive and negative conductive elements, and suitable distances (in inches) between inner and outer edges of the positive and negative conductive elements.

FIG. 4C is a bottom view of a wireless control unit with housing and positive and negative conductive elements, and suitable dimensions (in inches) of the housing.

FIG. 5 is a flow chart providing an embodiment of the wireless control unit operation via Bluetooth to produce electrostimulation. In the flow chart the conductive elements are labeled “conductive brackets.”

FIG. 6 displays an environmental view with the conductive kinesiology tape applied to the arm of a human being, with the tape having an elastic fabric and control unit.

FIG. 7 provides the cross-sectional side view of the conductive kinesiology tape of FIG. 3 engaged with the control unit of FIG. 4A.

### DETAILED DESCRIPTION

This description is intended only to acquaint others skilled in the art with the present conductive kinesiology tape, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application and may be variously modified.

In one embodiment, the conductive kinesiology tape includes an elastic fabric having a bottom surface and a top surface, with the top surface opposite the bottom surface. The conductive kinesiology tape includes conductive threads incorporated into the elastic fabric such that a portion of each conductive thread is exposed on the top surface of the elastic fabric, and a portion of each conductive thread is exposed on the bottom surface of the elastic fabric. The conductive kinesiology tape further includes an adhesive layer coupled to the bottom surface.

In another embodiment, the conductive kinesiology tape includes a first layer and a second layer. The first layer includes an clastic fabric having the bottom surface and the top surface. The first layer also includes a first conductive thread incorporated into the elastic fabric such that a portion of the first conductive thread is exposed on the top surface of the elastic fabric to form a positive conductive area, and a portion of the first conductive thread is exposed on the bottom surface of the elastic fabric to form a positive electrode. The first layer further includes a second conductive thread incorporated into the elastic fabric such that a portion of the second conductive thread is exposed on the top surface of the elastic fabric to form a negative conductive area, and a portion of the second conductive thread is exposed on the bottom surface of the elastic fabric to form a negative electrode. The second layer includes an adhesive disposed on the bottom surface of the elastic fabric. In other words, the adhesive is adhered to or otherwise attached to the bottom surface of the elastic fabric. In certain embodiments, the kinesiology tape consists essentially of or consists of the first and second layers.

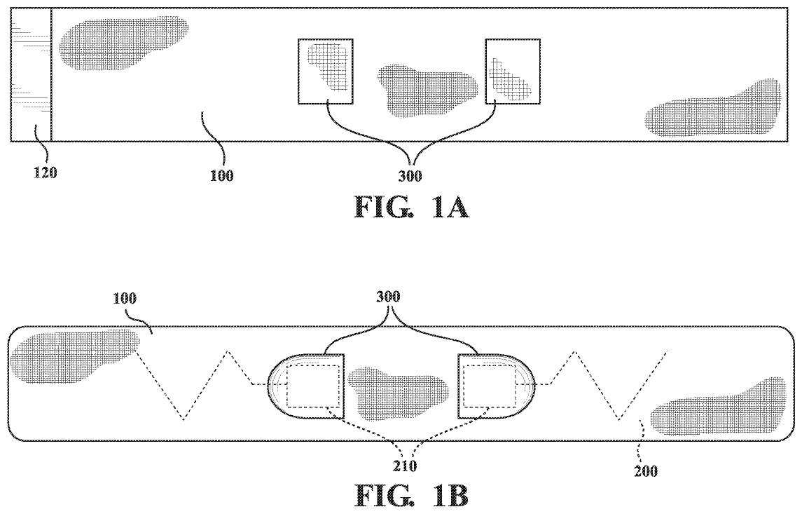

Referring now to the elastic fabric, any suitable elastic fabric may be used. In certain embodiments, the elastic fabric corresponds to the fabric layer of standard kinesiology tape in terms of design and function. In other words, in some embodiments the elastic fabric is made in the same way (e.g., woven or stitched), using the same materials (e.g., elastic cotton), and with the same design and dimensions (e.g., elongated strip, having thickness and elasticity similar to human skin) as the fabric layer of standard kinesiology tape. In certain embodiments, the elastic fabric is elongated. Elongated elastic fabric (100) is shown in FIG. 1A and FIG. 1B.

In certain embodiments, the elastic fabric comprises cotton. Cotton is advantageous in that it is soft, breathable, elastic, quick drying, and hypoallergenic. The elastic fabric may comprise 100% cotton or may comprise cotton blended with other fibers such as spandex, nylon, or polyester. In certain embodiments, the elastic fabric comprises 100% cotton. In certain embodiments, the elastic fabric comprises cotton blended with up to 10% spandex, such as 98% cotton/2% spandex or 95% cotton/5% spandex. In certain embodiments, the elastic fabric comprises cotton blended with up to 10% nylon, such as 98% cotton/2% nylon or 95% cotton/5% nylon. In certain embodiments, the elastic fabric comprises cotton blended with up to 90% polyester, such as 50% cotton/50% polyester or 60% cotton/40% polyester.

In certain embodiments, the elastic fabric comprises synthetic fibers. Synthetic fibers arc advantageous in that they are strong and may facilitate wearing of the tape for longer periods of time. In certain embodiments, the elastic fabric comprises rayon, optionally blended with another fiber such as spandex. In certain embodiments, the elastic fabric comprises rayon blended with up to 10% spandex, such as 98% rayon and 2% spandex or 95% rayon and 5% spandex. For the purpose of this disclosure, any reference to a percentage of a certain material in a layer represents the wt. % of the component based on the total weight of the layer.

The elastic fabric should have elastic properties similar to standard kinesiology tape. Like standard kinesiology tape, the elastic fabric will typically be longitudinally stretched by about 25% to about 50% prior to application. In certain embodiments, the elastic fabric can be longitudinally stretched by up to about 50%. In certain embodiments, the elastic fabric can be longitudinally stretched by up to about 60%. In certain embodiments, the elastic fabric can be longitudinally stretched by up to about 70%. In certain embodiments, the elastic fabric can be longitudinally stretched by up to about 80%. In certain embodiments, the elastic fabric can be longitudinally stretched by up to about 90%.

The elastic fabric has a bottom surface and a top surface. When the conductive kinesiology tape is applied to the skin of a subject, the bottom surface faces towards the subject's skin, and the top surface faces away from the subject's skin.

Referring now to the conduct thread, the conductive threads may be the same or different. In certain embodiments, the conductive threads are substantially the same, except that the first conductive thread forms the positive conductive area and the positive electrode, and the second conductive thread forms the negative conductive area and the negative electrode.

The first conductive thread is incorporated into the elastic fabric such that it forms a positive conductive area on the top surface of the elastic fabric and a positive electrode on the bottom surface of the elastic fabric. To enhance delivery of electrostimulation therapy and to provide maximum wearer safety and comfort, in certain embodiments the first conductive thread is exposed on the top surface of the elastic fabric only in the positive conductive area and on the bottom surface of the elastic fabric only at the positive electrode. In other words, in certain embodiments the portion of the first conductive thread between the positive conductive area and the positive electrode is not exposed on either surface of the elastic fabric. The portion of the first conductive thread between the positive conductive area and the positive electrode may be prevented from exposure to the top and bottom surfaces of the elastic fabric in any suitable manner. In certain embodiments, the portion of the first conductive thread between the positive conductive area and the positive electrode is wrapped in an insulating material such as cotton. In certain embodiments, the portion of the first conductive thread between the positive conductive area and the positive electrode may be shielded from exposure on the top and bottom surfaces of the elastic fabric by the elastic fabric itself. In other words, the first conductive thread may be incorporated within the elastic fabric such that the portion between the positive conductive area and the positive electrode is towards the axial center of the elastic fabric, with elastic fabric both above and below the first conductive thread.

The second conductive thread is incorporated into the elastic fabric such that it forms a negative conductive area on the top surface of the elastic fabric and a negative electrode on the bottom surface of the elastic fabric. To enhance delivery of electrostimulation therapy and to provide maximum wearer safety and comfort, in certain embodiments the second conductive thread is exposed on the top surface of the elastic fabric only in the negative conductive area and on the bottom surface of the elastic fabric only at the negative electrode. In other words, in certain embodiments the portion of the second conductive thread between the negative conductive area and the negative electrode is not exposed on either surface of the elastic fabric. The portion of the second conductive thread between the negative conductive area and the negative electrode may be prevented from exposure to the top and bottom surfaces of the elastic fabric in any suitable manner. In certain embodiments, the portion of the second conductive thread between the negative conductive area and the negative electrode is wrapped in an insulating material such as cotton. In certain embodiments, the portion of the second conductive thread between the negative conductive area and the negative electrode may be shielded from the top and bottom surfaces of the elastic fabric by the elastic fabric itself. In other words, the second conductive thread may be incorporated within the elastic fabric such that the portion between the negative conductive area and the negative electrode is towards the axial center of the elastic fabric, with elastic fabric both above and below the second conductive thread.

The conductive threads can be incorporated into the elastic fabric in any suitable manner. In certain embodiments, the conductive threads arc woven or stitched into the clastic fabric. In certain embodiments, the positive conductive area and the negative conductive area are located between the positive electrode and the negative electrode. In certain embodiments, the positive conductive area and the negative conductive area are located near the longitudinal center of the elastic fabric, the positive electrode is located between the positive conductive area and one end of the elastic fabric, and the negative electrode is located between the negative conductive area and the other end of the elastic fabric.

In certain embodiments, the conductive kinesiology tape is stretched in the longitudinal direction prior to application. In certain embodiments, the conductive threads do not negatively affect the elastic properties of the elastic fabric and are not themselves negatively affected by the stretching. In certain embodiments, the conductive threads stretch proportionally to the elastic fabric when the conductive kinesiology tape is stretched in the longitudinal direction prior to application.

To facilitate stretching, in certain embodiments the conductive threads are incorporated in the elastic fabric in a zigzag pattern. In other words, as the first conductive thread and second conductive thread extend longitudinally from the positive conductive area and negative conductive area, respectively, to the positive electrode and negative electrode, respectively, the first conductive thread and the second conductive thread extend back and forth toward and away from the lateral edges of the elastic fabric. Note in this regard that the zigzag pattern may comprise sudden changes of direction away from the lateral edge of the fabric such that the first conduct thread and/or the second conductive thread appear like a triangle wave. Alternatively, the conductive threads may extend longitudinally parallel to the lateral edge of the elastic fabric for a short distance before extending back towards the other edge, forming a square wave. Alternatively, the conductive threads may be incorporated such that there is a gradual change of direction toward and away from the lateral edges of the elastic fabric, like a sine wave. In certain embodiments, the conductive threads are incorporated into the elastic fabric in a triangle wave pattern.

The conductive threads can be made of any suitable conductive material. In certain embodiments, the conductive threads comprise at least one member independently chosen from carbon nanotubes, metallized thread, conductive polymer nanofibers, and reduced graphene. In certain embodiments, the conductive threads comprise at least one member independently chosen from carbon nanotubes and metallized thread. In certain embodiments, the conductive threads comprise silver thread. In certain embodiments, the conductive threads comprise carbon nanotubes. Carbon nanotubes are advantageous in that they are electrically conductive and possess exceptional tensile strength. In certain embodiments, the conductive threads comprise carbon nanotube yarn, such as carbon nanotube yarn made by plying, twisting, or braiding carbon nanotube individual fiber filaments together. An advantage of carbon nanotube yarn is that it has higher elasticity compared to individual carbon nanotube fiber filament. Carbon nanotube yarn and individual fiber filament suitable for the conductive threads are available commercially.

An advantage of the present conductive kinesiology tape is that the conductive threads, which permit the conductive kinesiology tape to be used for electrostimulation therapy, do not negatively impact its functioning as a traditional kinesiology tape. In certain embodiments, the elastic fabric containing the conductive threads continues to possess the elasticity, breathability, comfort, and function of normal kinesiology tape. To the wearer, it feels like normal kinesiology tape even though it is enhanced with the benefits of electrostimulation.

Referring now to the adhesive layer, the layer of adhesive is attached or applied to the bottom surface of the elastic fabric. In certain embodiments, a layer of adhesive is attached to the positive and negative electrodes. In certain embodiments, the adhesive layer attached to the bottom surface of the elastic fabric and optionally to the positive electrode and the negative electrode is a thin film. Any suitable adhesive may be used. In certain embodiments, the adhesive is a pressure-sensitive adhesive. In certain embodiments, the adhesive is a medical grade adhesive. In certain embodiments, the adhesive is flexible, elastic, breathable, non-irritating, and hypoallergenic. In certain embodiments, when the conductive kinesiology tape is stretched to expanded form prior to application the adhesive layer stretches proportionally to the elastic fabric. In certain embodiments, the adhesive meets ISO 10993 standards for skin sensitization and irritation. In certain embodiments, the adhesive maintains a high moisture vapor transmission rate (MVTR). In certain embodiments, the adhesive MVTR is as high or higher than the MVTR of human skin. In certain embodiments, the adhesive permits the elastic fabric and the positive and negative electrodes to be removed from the skin with a low-peel release force that does not cause damage or pain yet maintains tack and adhesion after several applications. Commercially available adhesives used on standard kinesiology tape are suitable for the present conductive kinesiology tapc. In certain embodiments, the adhesive comprises an acrylate adhesive. In certain embodiments, the adhesive comprises an acrylic acid polymer. In certain embodiments, the adhesive comprises a methacrylic acid polymer. In certain embodiments, the adhesive comprises an epoxy diacrylate polymer. In certain embodiments, the adhesive comprises a silicone adhesive.

In certain embodiments, the adhesive layer does not completely cover the elastic fabric. In certain embodiments, the adhesive layer does not completely cover the positive electrode and the negative electrode. Including gaps in adhesive coverage can improve breathability, reduce irritation, and reduce transmission of electrical impulses through the adhesive away from the skin beneath the electrodes. In certain embodiments, the adhesive layer comprises a wavelike pattern with alternating areas with and without adhesive.

The adhesive layer attached to the bottom surface of the elastic fabric can be the same or different from an adhesive layer attached to the positive electrode and the negative electrode. In certain embodiments, the adhesive layer attached to the bottom surface of the elastic fabric is the same as the adhesive layer attached to the positive electrode and the negative electrode. In certain embodiments, the adhesive layer attached to the bottom surface of the elastic fabric is different from the adhesive layer attached to the positive electrode and the negative electrode. In certain embodiments, the adhesive layer attached to the positive electrode and the negative electrode is conducting to facilitate the transmission of electrical impulses from the positive electrode to the skin and from the skin to the negative electrode. If the adhesive layer attached to the bottom surface of the elastic fabric is also conducting, the adhesive layer may comprise coverage gaps near the perimeter of the positive electrode and the negative electrode to prevent the electrical impulses dispersing throughout the adhesive layer and promote transmission of the electrical impulses through the skin in the area beneath the positive electrode and the negative electrode. In certain embodiments, the adhesive layer attached to the bottom surface of the elastic fabric is non-conducting. In certain embodiments, the adhesive layer attached to the positive electrode and the negative electrode comprises additives to enhance electrical conductivity. In certain embodiments, the adhesive layer attached to the bottom surface of the elastic fabric comprises additives to enhance electrical conductivity. In certain embodiments, the adhesive layer attached to the bottom surface of the elastic fabric and/or to the positive electrode and negative electrode comprises one or more additives chosen from chitosan-aniline, metal particles, such as copper, aluminum, or silver particles, electroconductive carbon black, carbon fibers, metalized glass spheres, and metalized glass fibers. In certain embodiments, the adhesive layer attached to the bottom surface of the elastic fabric and/or to the positive electrode and negative electrode comprises one or more additives chosen from chitosan-aniline, electroconductive carbon black, and carbon fibers.

Traditional TENS and EMS devices require the electrodes to be covered with a conductive hydrogel to attach the electrodes and promote transmission of electrical impulses through the skin. Such aqueous hydrogels are disadvantageous in that during storage they may lose water and experience a deterioration of properties if not sealed in an air-tight manner. They can also be messy, slimy, and unpleasant against the skin. An advantage of the present conductive kinesiology tape is that the adhesive layer attached to the positive electrode and the negative electrode need not comprise a hydrogel. This simplifies storage, extends shelf life, and renders the conductive kinesiology tape more pleasant to wear. In certain embodiments, the adhesive layer attached to the bottom surface of the elastic fabric does not comprise a hydrogel. In certain embodiments, the adhesive layer attached to the positive electrode and the negative electrode does not comprise a hydrogel. In certain embodiments, the adhesive layer attached to the bottom surface of the elastic fabric and to the positive electrode and the negative electrode does not comprise a hydrogel.

In certain embodiments, the adhesive layer attached to the bottom surface of the elastic fabric further comprises a therapeutic agent suitable for transdermal or topical administration. In certain embodiments, the adhesive layer attached to the positive electrode and the negative electrode further comprises a therapeutic agent suitable for transdermal or topical administration. In certain embodiments, the adhesive layer attached to the bottom surface of the elastic fabric and/or to the positive electrode and the negative electrode comprises a sustained-release therapeutic agent. In certain embodiments, the therapeutic agent comprises one or more substances chosen from vitamins, analgesics, anesthetics, antipruritic agents, anti-inflammatory agents, and cannabis oils. In certain embodiments, the therapeutic agent comprises one or more substances chosen from lidocaine, benzocaine, vitamin A, ascorbic acid, vitamin B, biotin, pantothenic acid, vitamin D, vitamin E, menthol, methyl salicylate, and cannabidiol. In certain embodiments, the therapeutic agent comprises one or more substances chosen from menthol, methyl salicylate, and cannabidiol.

In certain embodiments, the adhesive layer is covered with a removable masking layer. It is to be appreciated that when the conductive kinesiology tape consists essentially of the first and second layers, the removable masking layer is within the scope of such a statement. The removable masking layer prevents the adhesive layer from sticking to itself or to other objects prior to application. Typically, the removable masking layer is removed and disposed of to expose the adhesive layer prior to use. In certain embodiments, the removable masking layer is easily released from the adhesive layer. Any suitable removable masking layer may be used. In certain embodiments, the removable masking layer may comprise the container in which the conductive kinesiology tape is packaged for sale. In such embodiments, the conductive kinesiology tape may be peeled from the container prior to use. In certain embodiments, the removable masking layer comprises at least one material chosen from paper and a polymer film. In certain embodiments, the polymer film is high-density polyethylene. In certain embodiments, the removable masking layer comprises paper and the adhesive layer comprises an acrylic polymer. In certain embodiments, the removable masking layer comprises high-density polyethylene and the adhesive layer comprises silicone. In certain embodiments, the removable masking layer comprises paper. In certain embodiments, the removable masking layer comprises high-density polyethylene. FIG. 1A shows the conductive kinesiology tape attached to a removable masking layer (120).

In certain embodiments, a control unit comprising a positive conductive element and a negative conductive element is attached to the conductive kinesiology tape to provide electrostimulation. In certain embodiments, the control unit is attached to the conductive kinesiology tape such that the positive conductive element is in conductive contact with the positive conductive area and a negative conductive element is in conductive contact with the negative conductive area. In certain embodiments, the control unit comprises a) a housing, b) a positive conductive element extending from the housing and adapted to make conductive contact with the positive conductive area; c) a negative conductive element extending from the housing and adapted to make conductive contact with the negative conductive area; d) a power source; and e) control circuitry. In certain embodiments, the control unit generates low voltage electrical impulses adapted for transcutaneous electrical nerve stimulation or electrical muscle stimulation. In certain embodiments, the control unit generates low voltage electrical impulses for transmission from the positive conductive element to the positive conductive area and then the positive electrode to provide electrostimulation of nerve or muscle fibers located between the positive electrode and the negative electrode.

Any suitable control unit may be used. In certain embodiments, the control unit comprises a handheld or tabletop TENS or EMS electrical impulse generating device, wherein the positive conductive element and the negative conductive element comprise wires extending from the device to the positive conductive area and the negative conductive area. In certain embodiments, the control unit is a wireless control unit that can be removably attached to the conductive kinesiology tape comprising a) a housing, b) a positive conductive element extending from the housing and adapted to make conductive contact with the positive conductive area; c) a negative conductive element extending from the housing and adapted to make conductive contact with the negative conductive area; d) a power source; and e) control circuitry.

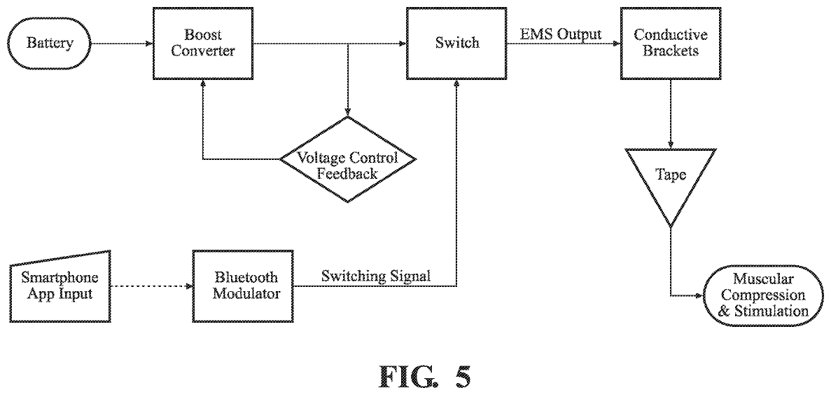

Referring now to FIG. 5, in certain embodiments, the wireless control unit comprises a battery. In certain embodiments, the battery is rechargeable. In certain embodiments, the battery is a lithium rechargeable battery. In certain embodiments, the battery is a 3.7 volt, 600 mAh lithium rechargeable battery. In certain embodiments, the battery is located in the housing of the wireless control unit.

In certain embodiments, the control circuitry provides an electric signal with adjustable pulse intensity, frequency, and duty cycle. In certain embodiments, the control circuitry comprises an integrated circuit chip. In certain embodiments, the control circuitry is located in the housing of the control unit.

With continued reference to FIG. 5, in certain embodiments, the wireless control unit comprises a boost converter and a voltage feedback control loop. In certain embodiments, the battery voltage is initially stepped up and current is stepped down by a boost converter. In certain embodiments, these values are maintained through the voltage control feedback loop. In certain embodiments, the voltage control feedback loop is built into the integrated circuit chip. In certain embodiments, the boost converter and the voltage feedback loop are located in the housing of the wireless control unit.

In certain embodiments, the wireless control unit comprises a Bluetooth antenna and a transistor switch. In certain embodiments, the electric signal output is dependent on the transistor switch, which is driven by a switching signal sent to the Bluetooth antenna from a Bluetooth device such as a phone or tablet. In certain embodiments, using an app on the Bluetooth device, the subject can adjust the frequency, duty cycle, or intensity of the electric pulse. In certain embodiments, the Bluetooth antenna and transistor switch are located in the housing of the wireless control unit.

In certain embodiments, the wireless control unit comprises a rechargeable battery, a voltage protection feedback loop, and a Bluetooth antenna. In certain embodiments, the rechargeable battery, the voltage protection feedback loop, and the Bluetooth antenna are located in the housing of the wireless control unit.

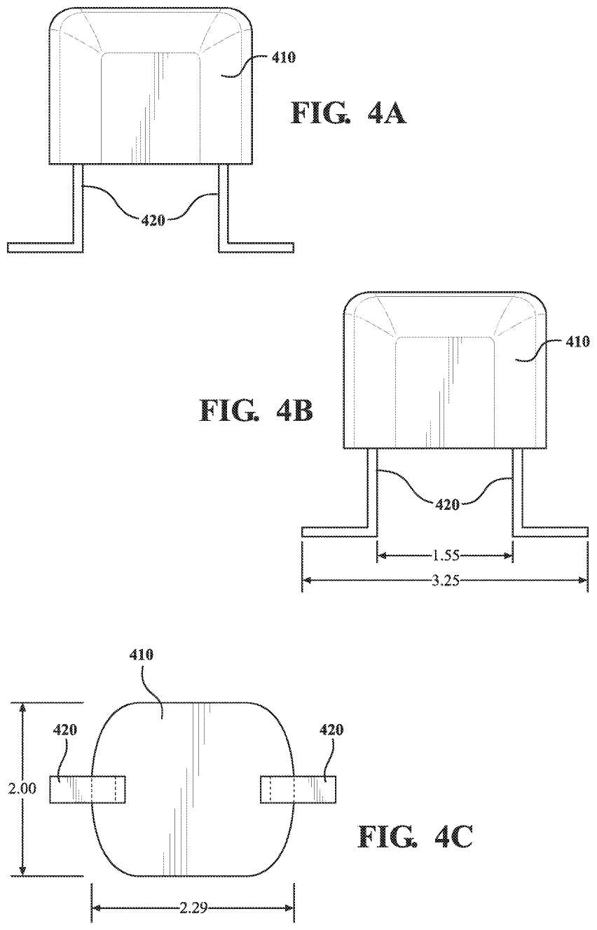

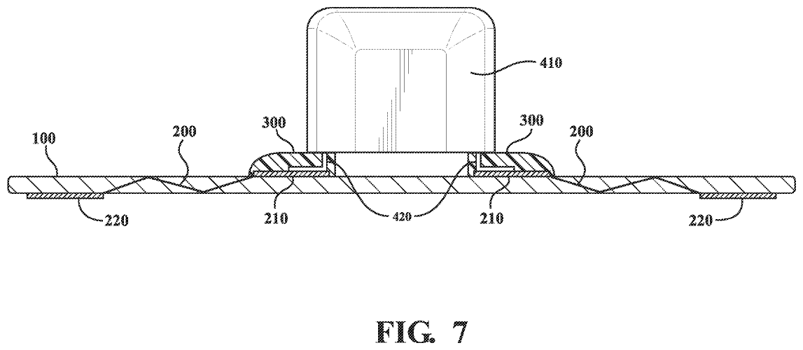

In certain embodiments, the wireless control unit comprises a positive conductive element adapted for conductive contact with the positive conductive area and a negative conductive element adapted for conductive contact with the negative conductive area. In certain embodiments, the negative conductive element and the negative electrode are grounded on the same copper plane as the battery of the wireless control unit. The positive conductive element and the negative conductive element may comprise any suitable conductive material. In certain embodiments, the positive conductive element and the negative conductive element comprise a conductive metal such as copper. In certain embodiments, the positive conductive element and the negative conductive element are adapted to removably attach the wireless control unit to the conductive kinesiology tape. In certain embodiments, the positive conductive element and the negative conductive element snap onto the conductive kinesiology tape. In certain embodiments, the positive conductive element and the negative conductive element snap onto the conductive kinesiology tape at the positive conductive area and the negative conductive area. In certain embodiments, the top surface of the elastic fabric comprises two pockets situated above the positive conductive area and the negative conductive area. In certain embodiments, the top surface of the elastic fabric comprises two pockets situated above the positive conductive area and the negative conductive area adapted to receive and securely hold the positive conductive element and the negative conductive element of the control unit. FIG. 1 and FIG. 3 show pockets (300) on the top surface of the conductive kinesiology tape. In certain embodiments, the positive conductive element and the negative conductive element slide into pockets on the top surface of the clastic fabric situated above the positive conductive area and the negative conductive area, and the positive conductive element and the negative conductive element are securely held in contact with the positive conductive area and the negative conductive area, respectively, within the pockets. In certain embodiments, the housing of the wireless control unit comprises springs that force the positive conductive element and the negative conductive element against the sides of the housing. In certain embodiments, the housing of the wireless control unit comprises springs that force the positive conductive element and the negative conductive element against the sides of the housing, permit the positive conductive element and the negative conductive element to move towards one another when pressed, and cause the positive conductive element and the negative conductive element to move away from one another towards the sides of the housing when released. In certain embodiments, the housing of the wireless control unit comprises springs that force the positive conductive element and the negative conductive element against the sides of the housing, permit the positive conductive element and the negative conductive element to move towards one another when pressed, cause the positive conductive element and the negative conductive element to move away from one another towards the sides of the housing when released, and are adapted to permit the positive conductive element and the negative conductive element to be removably inserted into and securely held by pockets on the top surface of the elastic fabric above the positive conductive area and the negative conductive area, respectively. In certain embodiments, the positive conductive element and the negative conductive element are spring loaded in the wireless control unit such that a subject may squeeze them together to permit loading into the pockets, and when released spring back away from each other and are securely held in the pockets. FIG. 4 depicts several views of a control unit with positive and negative conductive elements (420) adapted to be squeezed together to permit attachment to the conductive kinesiology tape. For example, as shown in FIG. 7, the conductive elements (420) may be squeezed to place the conductive elements (420) in the pockets (300) and in contact with the conductive areas (210). In certain embodiments, the positive conductive area and the negative conductive area are located between the positive electrode and the negative electrode, and the wireless control unit is attached to the conductive kinesiology tape between the positive electrode and the negative electrode such that the positive conductive element and the negative conductive element are in conductive contact with the positive conductive area and the negative conductive area, respectively. In certain embodiments, the positive conductive area and the negative conductive area are located near the longitudinal center of the elastic fabric, the wireless control unit is attached to the conductive kinesiology tape near the longitudinal center of the elastic fabric such that the positive conductive element and the negative conductive element are in conductive contact with the positive conductive area and the negative conductive area, respectively, the positive electrode is located between the positive conductive area and one end of the elastic fabric, and the negative electrode is located between the negative conductive area and the other end of the elastic fabric.

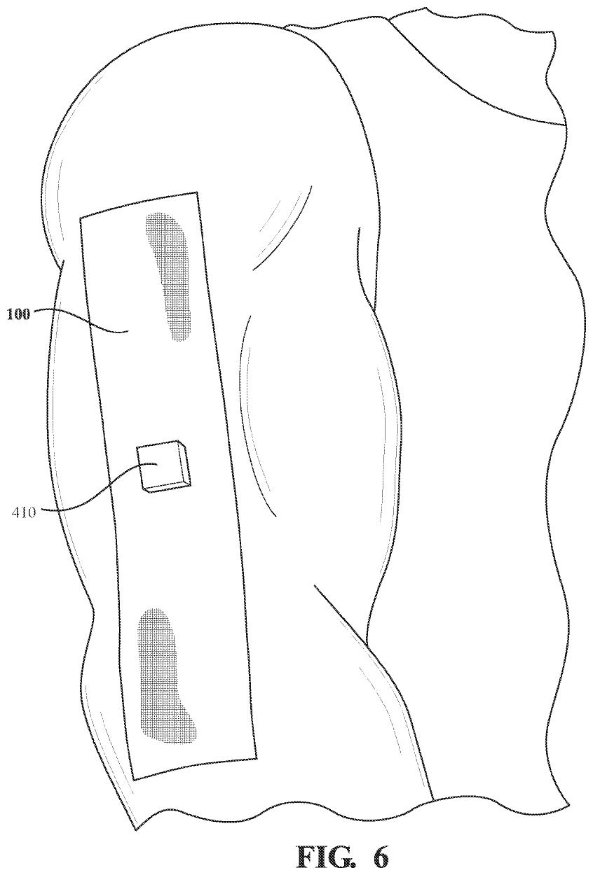

Referring now to FIG. 6, the conductive kinesiology tape has many uses. In certain embodiments, there is provided a method of enhancing muscle regeneration in a subject in need thereof using the conductive kinesiology tape described herein, comprising the steps of: stretching the conductive kinesiology tape by about 10% to about 80%, applying the stretched conductive kinesiology tape to the subject above the muscle in need of enhanced regeneration, attaching the positive conductive element and the negative conductive element of the control unit described herein to the positive conductive area and the negative conductive area, respectively, of the applied conductive kinesiology tape, and applying electrostimulation to the muscle.

In certain embodiments, there is provided a method of activating muscles to increase strength or recovery in a subject in need thereof using the conductive kinesiology tape described herein, comprising the steps of: stretching the conductive kinesiology tape by about 10% to about 80%, applying the stretched conductive kinesiology tape to the subject above the muscle in need of activation, attaching the positive conductive element and the negative conductive element of the control unit described herein to the positive conductive area and the negative conductive area, respectively, of the applied conductive kinesiology tape, and applying electrostimulation to the muscle.

In certain embodiments, there is provided a method of relieving pain in a subject in need thereof using the conductive kinesiology tape described herein, comprising the steps of: stretching the conductive kinesiology tape by about 10% to about 80%, applying the stretched conductive kinesiology tape to the subject above the painful area, attaching the positive conductive element and the negative conductive element of the control unit described herein to the positive conductive area and the negative conductive area, respectively, of the applied conductive kinesiology tape, and applying electrostimulation to the painful area. In certain embodiments, there is provided a method of relieving arthritis joint pain in a subject in need thereof using the conductive kinesiology tape described herein, comprising the steps of: stretching the conductive kinesiology tape by about 10% to about 80%, applying the stretched conductive kinesiology tape to the subject above the painful joint, attaching the positive conductive element and the negative conductive element of the control unit described herein to the positive conductive area and the negative conductive area, respectively, of the applied conductive kinesiology tape, and applying electrostimulation to the joint.

In certain embodiments, the subject is a mammal. In certain embodiments, the subject is a human being. In certain embodiments, the stretched conductive kinesiology tape is applied to the skin of the subject.

In certain embodiments, the conductive kinesiology tape is stretched by about 10% to about 70% before application to the subject. In certain embodiments, the conductive kinesiology tape is stretched by about 10% to about 60% before application to the subject. In certain embodiments, the conductive kinesiology tape is stretched by about 20% to about 80% before application to the subject. In certain embodiments, the conductive kinesiology tape is stretched by about 20% to about 70% before application to the subject. In certain embodiments, the conductive kinesiology tape is stretched by about 20% to about 60% before application to the subject. In certain embodiments, the conductive kinesiology tape is stretched by about 30% to about 80% before application to the subject. In certain embodiments, the conductive kinesiology tape is stretched by about 30% to about 70% before application to the subject. In certain embodiments, the conductive kinesiology tape is stretched by about 20% to about 60% before application to the subject. In certain embodiments, the conductive kinesiology tape is stretched by about 30% to about 60% before application to the subject. In certain embodiments, the conductive kinesiology tape is stretched by about 40% to about 80% before application to the subject. In certain embodiments, the conductive kinesiology tape is stretched by about 40% to about 70% before application to the subject. In certain embodiments, the conductive kinesiology tape is stretched by about 40% to about 60% before application to the subject. In certain embodiments, the conductive kinesiology tape is stretched by about 10% before application to the subject. In certain embodiments, the conductive kinesiology tape is stretched by about 20% before application to the subject. In certain embodiments, the conductive kinesiology tape is stretched by about 30% before application to the subject. In certain embodiments, the conductive kinesiology tape is stretched by about 40% before application to the subject. In certain embodiments, the conductive kinesiology tape is stretched by about 50% before application to the subject. In certain embodiments, the conductive kinesiology tape is stretched by about 60% before application to the subject. In certain embodiments, the conductive kinesiology tape is stretched by about 70% before application to the subject. In certain embodiments, the conductive kinesiology tape is stretched by about 80% before application to the subject.

### Examples

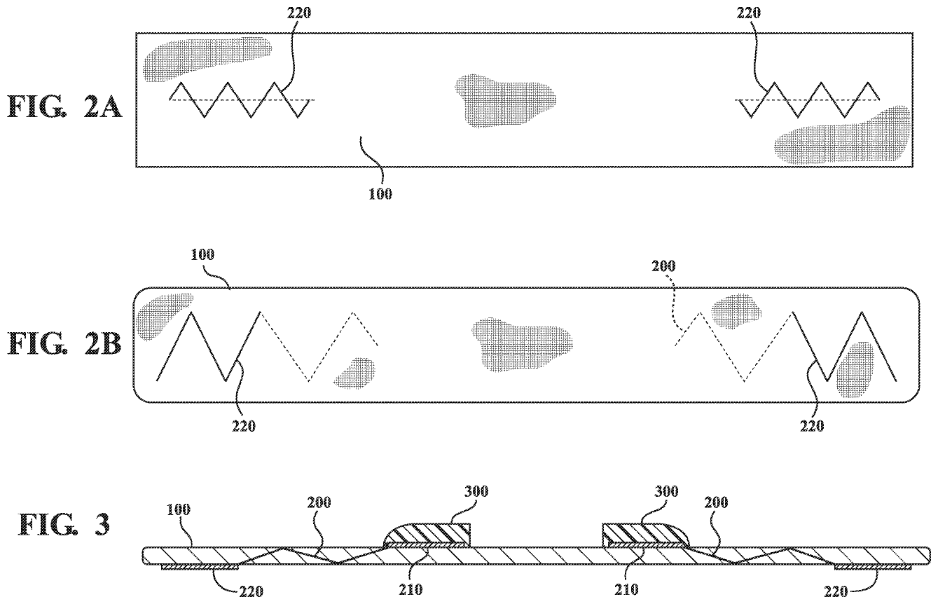

FIGS. 1A, 1B, 2A, and 2B show the conductive kinesiology tape. The tape is woven in an interwoven pattern first on the far left exposed on the bottom surface of the elastic fabric (100) as a threaded electrode (220), and then is woven into the middle of the elastic fabric as to no longer expose the electrode and then externally exposed on the top surface of the elastic fabric beneath the pocket (300) to mate to the control unit. A second thread is mirrored in the same design on the other side.

FIG. 3 shows a cross sectional drawing of the conductive kinesiology tape. From left to right it is shown that the conductive thread (200) first being exposed on the underside of the elastic fabric (100) to form an electrode (220), then is interwoven through the middle of the elastic fabric in a zig-zag pattern finally being exposed externally on the top surface of the elastic fabric to form a conductive area (210) to be mated to the control unit. The threading is reciprocated on the other side of the elastic fabric.

FIGS. 4A, 4B, and 4C show a wireless control unit (410) that can be mated into the pockets of the conductive kinesiology tape (100) and be freely disconnected from the tape by squeezing together the conductive elements (420). In certain embodiments, such as shown in FIG. 6, the control unit (410) envelopes or covers the pockets (300). In other embodiments, such as shown in FIG. 7, the control unit (410) may partially cover the pockets (300).

1. A conductive kinesiology tape comprising:
a. an elastic fabric having a bottom surface and a top surface;
b. conductive threads incorporated into the elastic fabric such that a portion of each conductive thread is exposed on the top surface of the elastic fabric, and a portion of each conductive thread is exposed on the bottom surface of the elastic fabric;
c. an adhesive layer coupled to the bottom surface; and
d. a control unit comprising:
i. a housing,
ii. a positive conductive element extending from the housing and adapted to make conductive contact with a first conductive thread on the top surface of the elastic fabric,
iii. a negative conductive element extending from the housing and adapted to make conductive contact with a second conductive thread on the top surface of the elastic fabric,
iv. a power source, and
v. control circuitry.

2. (canceled)

3. The conductive kinesiology tape of claim 1, wherein the housing further comprises springs that force the positive conductive element and the negative conductive element against at least one side of the housing.

4. The conductive kinesiology tape of claim 1, wherein the elastic fabric comprises pockets adapted to receive and securely hold the positive conductive element and the negative conductive element of the control unit.

5. A conductive kinesiology tape comprising:
a. a first layer comprising:
i. an elastic fabric having a bottom surface and a top surface,
ii. a first conductive thread incorporated into the elastic fabric such that
1. a portion of the first conductive thread is exposed on the top surface of the elastic fabric to form a positive conductive area, and
2. a portion of the first conductive thread is exposed on the bottom surface of the elastic fabric to form a positive electrode; and
iii. a second conductive thread incorporated into the elastic fabric such that
1. a portion of the second conductive thread is exposed on the top surface of the elastic fabric to form a negative conductive area, and
2. a portion of the second conductive thread is exposed on the bottom surface of the elastic fabric to form a negative electrode; and
b. a second layer comprising an adhesive attached to the bottom surface of the elastic fabric.

6. The conductive kinesiology tape as set forth in claim 5, wherein the elastic fabric comprises elastic cotton and nylon.

7. The conductive kinesiology tape as set forth in claim 5, wherein the conductive threads are incorporated into the elastic fabric in a zigzag pattern.

8. The conductive kinesiology tape as set forth in claim 5, wherein the conductive threads are woven or stitched into the elastic fabric.

9. The conductive kinesiology tape as set forth in claim 5, wherein the conductive threads comprise at least one member chosen from carbon nanotubes, metallized thread, conductive polymer nanofibers, and reduced graphene.

10. The conductive kinesiology tape as set forth in claim 9, wherein the conductive threads comprise carbon nanotubes.

11. The conductive kinesiology tape as set forth in claim 9, wherein the conductive threads comprise metallized thread.

12. (canceled)

13. The conductive kinesiology tape as set forth in claim 5, wherein the adhesive comprises an acrylic polymer.

14. The conductive kinesiology tape as set forth in claim 5, wherein adhesive comprises a methacrylate polymer or epoxy diacrylate polymer.

15. The conductive kinesiology tape as set forth in claim 5, wherein the adhesive further comprises a therapeutic agent.

16. (canceled)

17. The conductive kinesiology tape as set forth in claim 15, wherein the therapeutic agent comprises at least one member chosen from menthol, methyl salicylate, and cannabidiol.

18. (canceled)

19. The conductive kinesiology tape as set forth in claim 5, wherein the conductive kinesiology tape consists of the first and second layers and an optional removable masking layer disposed on the adhesive layer.

20. The conductive kinesiology tape as set forth in claim 5, wherein the conductive kinesiology tape is free of conductive ink.

21. The conductive kinesiology tape of as set forth in claim 5, further comprising a control unit, the control unit comprising:
a. a housing;
b. a positive conductive element extending from the housing and adapted to make conductive contact with the positive conductive area;
c. a negative conductive element extending from the housing and adapted to make conductive contact with the negative conductive area;
d. a power source; and
e. control circuitry.

22. The conductive kinesiology tape as set forth in claim 21, wherein the control unit generates low voltage electrical impulses adapted for transcutaneous electrical nerve stimulation or electrical muscle stimulation.

23. The conductive kinesiology tape as set forth in claim 21, wherein the control unit is wireless.

24. The conductive kinesiology tape as set forth in claim 23, wherein the control unit comprises a rechargeable battery, voltage protection feedback loop, and Bluetooth antenna.

25.-32. (canceled)
